(19) United States
(12) Patent Application Publication
(10) Pub. No.: US 2007/0232827 A1
(43) Pub. Date: Oct. 4, 2007

(54) Isocyanate Production Method

(75) Inventors: Andreas Wolfert, Bad Rappenau (DE); Hans-Jurgen Pallasch, Ludwigshafen (DE); Eckhard Stroefer, Mannheim (DE); Ulrich Penzel, Tettau (DE); Filip Deberdt, Muizen (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: BASF Aktiengesellschaft, Ludwigshafen (DE)

(21) Appl. No.: 11/569,445

(22) PCT Filed: May 21, 2002
(86) PCT No.: PCT/EP05/05520

§ 371 Date: Nov. 21, 2006

(30) Foreign Application Priority Data
May 25, 2004 (DE) 10 2004 026 095.8

(51) Int. Cl. C07C 263/10

(52) U.S. Cl. 560/347

(57) ABSTRACT

A process for the preparation of isocyanates by reacting amines with phosgene, the phosgene-containing stream of starting materials (i) being substantially free of isocyanates and (ii) having a mass content of hydrogen chloride (referred to below as HCl) of less than 0.4% by mass, and a production plant for carrying out the process according to the invention.

A process for the preparation of isocyanates by reacting amines with phosgene, the phosgene-containing stream of starting materials
(i) being substantially free of isocyanates and
(ii) having a mass content of hydrogen chloride (referred to below as HCl) of less than 0.4% by mass, and a production plant for carrying out the process according to the invention.

The literature has described various processes for the preparation of isocyanates by reacting amines with phosgene.

EP-A-322 647 describes the continuous preparation of mono- or polyisocyanates in a combination of a mixing nozzle in which the amine stream and the phosgene stream are thoroughly mixed and a downstream vertical tubular reactor which is cascaded by perforated trays. A disadvantage of this process is the tendency of blockage in the mixing nozzle, which reduces the operating stability of the process.

Thus, EP 0830 894 therefore describes the use of cleaning pins in the mixing apparatus in order to improve the operability of the mixing nozzle. A disadvantage is the use of moving parts in the mixing apparatus. At the passages, there is the risk of an undesired emergence of the toxic phosgene.

Further approaches regarding the optimization of the process consist in optimizing the streams of starting materials used, in order to improve the process with regard to the space-time yield.

WO 96/16028 describes a continuous process for the preparation of isocyanates, comprising a one-stage reaction with regard to the temperature and the use of isocyanate as a solvent for the phosgene, the chlorine content of the isocyanate being less than 2%. A tubular reactor can be used for the phosgenation. A disadvantage of the process is that the isocyanate is recycled continuously into the reaction zone, where it can react in the presence of the free amine to give ureas, which are precipitated as a solid. However, the stable operation of such a process is endangered by the problems of solids. Owing to the large amount of isocyanate circulated, there is a relatively large reaction volume, which is associated with an undesirably high cost in terms of apparatus.

U.S. Pat. No. 4,581,174 describes the continuous preparation of organic mono- and/or polyisocyanates by phosgenation of the primary amine in a mixing circulation with partial recycling of the isocyanate-containing reaction mixture, the proportion of HCl in the recycled mixture being less than 0.5%. Here too, it is true that the continuous recycling of the isocyanate into the reaction zone with free amine promotes the formation of urea. The precipitated urea endangers the stable operation of the process

GB 737 442 describes the recovery of phosgene from the isocyanate synthesis. The phosgene recovered has an HCl content of from 0.5 to 0.7%.

It is furthermore known that the use of a large excess of phosgene relative to the amine used leads to high selectivities with respect to the isocyanate prepared and therefore has a decisive influence on the cost-efficiency of the preparation process. With increasing ratio of phosgene to amino groups, there is a tendency for the phosgene hold-up of the plant and the volume of the plant to increase. On the other hand, owing to the toxicity of phosgene, a very short phosgene hold-up and a very compact design of the plant are desirable This simultaneously represents a reduction in the capital costs of the plant and hence an improvement in the cost-efficiency of the process.

A further aspect for achieving good cost-efficiency is a long on-stream time of the plant without shutdown of the plant. Shutdowns are usually necessary in the isocyanate production plants when plant components become blocked by the solids forming during the phosgenation.

An object of the invention was therefore to provide a process for the preparation of isocyanates which makes it possible to carry out the resulting reactions with high selectivity and high space-time yield and high operating stability so that the process can be designed to be spatially compact and can be operated economically.

In particular, it was an object of the invention to provide a process for the preparation of isocyanates which permits a low level of solid formation and solid deposition in the production plant, in particular in the mixing nozzle and in the residence time reactor.

It has now been found that the process can be operated particularly effectively with respect to its operability if, firstly, the recycling of isocyanates frequently described in the prior art or the use of isocyanates as solvent is dispensed with and, secondly the HCl content in the phosgene-containing stream fed to the mixing apparatus or reaction apparatus is kept low.

The invention therefore relates to a process for the preparation of isocyanates by reacting amines with phosgene, the phosgene-containing stream of starting materials
(i) being substantially free of isocyanates and
(ii) having a mass content of hydrogen chloride of less than 0.4% by mass.

Furthermore, the invention relates to the use of phosgene, the phosgene being substantially free of isocyanates and having a mass content of hydrogen chloride of less than 0.4% by mass, for the preparation of isocyanates by phosgenation of primary amines.

Finally, the invention relates to a production plant for the preparation of isocyanates by reacting primary amines with phosgene, comprising an amine vessel, a phosgene vessel, a mixing apparatus, a reactor and a working-up apparatus, wherein the phosgene present in the phosgene vessel
(i) has a mass content of isocyanate of from 0.00001 to 1% and
(ii) a mass content of hydrogen chloride of from 0.00001% to less than 0.4% by mass.

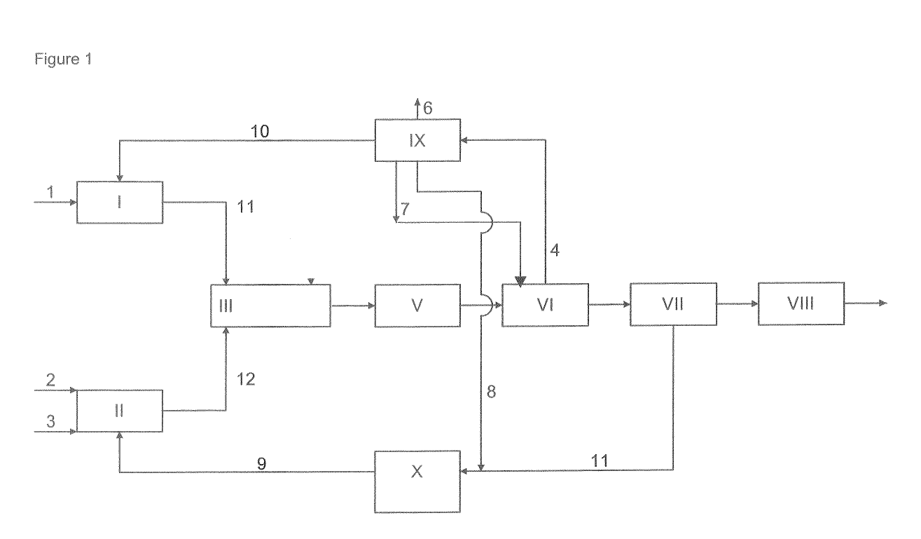

A preferred embodiment of the process according to the invention is illustrated in FIG. 1. In FIG. 1, the meanings are as follows:

I Phosgene vessel

II Amine vessel

III Mixing apparatus

V Reactor

VI First processing apparatus

VII Second processing apparatus (optional)

VIII Isocyanate vessel

IX Phosgene working-up

X Solvent working-up (optional)

1 Feed of fresh phosgene

2 Feed of fresh amine

3 Feed of inert solvent (optional)

4 Hydrogen chloride, phosgene, inert solvent and optionally small amounts of isocyanate which have been separated off

6 Hydrogen chloride discharged

7 Isocyanate separated off (optional)

8, 11 Inert solvent separated off (optional)

9 Inert solvent worked up (optional)

10 Phosgene worked up

11 Phosgene-containing stream of starting materials which has the features (i) and (ii) according to the invention

12 Amine-containing stream of starting materials

The amine from the amine vessel II and the phosgene from the phosgene vessel I are mixed in a suitable mixing apparatus III. The phosgene vessel I can be filled with fresh phosgene 1 or with recycled and worked-up phosgene 10. The material stream transferred from the phosgene vessel I into the mixing apparatus III is the phosgene-containing stream 11 of starting materials which has the features (i) and (ii) according to the invention.

After the mixing, the mixture is transferred to a reactor V. It is also possible to use apparatuses which constitute both a mixing apparatus and a reaction apparatus (i.e. III and V are combined into one apparatus), for example tubular reactors having flange-connected nozzles.

In the processing apparatus VI, hydrogen chloride and, if appropriate inert solvent and/or small proportions of the isocyanate stream are usually separated off from the isocyanate stream In the optional processing apparatus VII, inert solvent is preferably separated off, then worked up in a suitable apparatus X and recycled to the amine vessel II. For example the processing apparatuses may be conventional distillation units.

It is essential to the invention that the phosgene fed in and required for the reaction (=phosgene-containing stream 11 of starting materials which is fed in) have a mass content of hydrogen chloride of less than 0.4% by mass (ii). The phosgene-containing stream of starting materials preferably has a mass content of hydrogen chloride of from 0.00001% to less than 0.4% by mass, more preferably from 0.0001% to less than 0.3% by mass, particularly preferably from 0.0005% to less than 0.25% by mass and very particularly preferably from 0.001% to less than 0.2% by mass.

In the context of this invention the information in percentages by mass of the phosgene-containing stream of starting materials (features (i) and (ii)) is based on the total mass of the sum of phosgene, HCl and, if appropriate impurities of isocyanate. This information in percentages by mass is not based on the mass of the phosgene-containing stream of starting materials, including solvent, if the phosgene-containing stream of starting materials which is fed to the reaction or mixing apparatus additionally contains one or more solvents.

The required amount of HCl in the phosgene-containing stream 11 of starting materials can be provided by addition of HCl to the fresh phosgene or preferably by corresponding working-up of the phosgene stream 10 (i.e. the phosgene working-up IX is adjusted so that—taking into account the amount of fresh phosgene 1 fed in—the phosgene stream 10 provides the amount, according to the invention of HCl (ii) in the stream 11).

Furthermore, it is essential to the invention that the phosgene-containing stream of starting material contains substantially no isocyanates (i). This is to be understood as meaning that, in the process according to the invention no isocyanates prepared in the reactor (or other isocyanate compounds) are recycled and fed to the phosgene-containing stream of starting materials, or that isocyanates are used as solvents and are fed to the phosgene-containing stream of starting materials.

In the working-up apparatus VI, HCl, phosgene and, if appropriate, inert solvent and also small amounts of isocyanate are separated off from the main product stream (the isocyanate to be prepared). The isolation of isocyanate in the apparatus VI usually takes place for technical reasons but is not desired. Furthermore, isocyanate is in general again separated off 7 in the phosgene working-up IX, and said isocyanate can be fed, for example, to the first working-up apparatus VI. However, for technical reasons, the working-up apparatus VI may not provide complete, i.e. 1 00%, isolation of isocyanate.

In the context of this invention, it is important that the phosgene-containing stream of starting materials contain substantially no isocyanates (i). Nevertheless, as explained above, the phosgene-containing stream of starting materials may contain isocyanates in small amounts, for example because it is not possible technically to obtain the stream 10 completely free of isocyanates. “Substantially no isocyanates” is therefore to be understood as meaning that the phosgene-containing stream of starting materials usually contains less than 1% by weight, preferably from 0.00001% to less than 1% by mass, more preferably from 0.0001% to less than 0.5% by mass, even more preferably from 0.001% to less than 0.3% by mass, particularly preferably from 0.01% to less than 0.2% by mass, of isocyanates.

In the context of these inventions, “isocyanates” are understood as meaning all compounds which have at least one free isocyanate group.

For calculating the amount of HCl and isocyanates in the phosgene-containing stream of starting materials, carbamoyl chlorides are, if appropriate, also included. Carbamoyl chloride is formed by reaction of isocyanates with HCl in an equilibrium reaction. If the phosgene-containing stream of starting materials contains carbamoyl chloride, these are “theoretically” cleaved into HCl and phosgene, and the respective amount of the cleavage products is included in the calculation of the amounts of isocyanate (i) and HCl (ii).

It is furthermore preferable if the phosgene stream which is fed to the mixing of amine stream and phosgene stream already contains the abovementioned amount of HCl. The amount of HCl should not, as described in U.S. Pat. No. 3,234,253, only be introduced subsequently in the reaction mixture of amine and phosgene.

In the process according to the invention, the mixing of the reactants is effected in a mixing apparatus which is distinguished by high shearing of the reaction stream fed through the mixing apparatus. A preferably used mixing apparatus is a rotary mixing apparatus, a mixing pump or a mixing nozzle, which is installed upstream of the reactor. A mixing nozzle is particularly preferably used. The mixing time in this mixing apparatus is usually from 0.0001 s to 5 s, preferably from 0.0005 to 4 s, particularly preferably from 0.001 to 3 s. The mixing time is to be understood as meaning the time which elapses from the beginning of the mixing process until 97.5% of the fluid elements of the mixture obtained have a mixing fraction which, based on the value of the theoretical end value of the mixing fraction of the mixture obtained on reaching the state of perfect mixing, deviates less than 2.5% from this end value of the mixing fraction (for the concept of the mixing fraction, cf. for example J. Warnatz, U. Maas, R. W, Dibble: Verbrennung, Springer Verlag, Berlin Heidelberg New York. 1997, 2nd Edition, page 134).

In a preferred embodiment, the reaction of amine with phosgene is effected at absolute pressures of from 0.9 bar to 400 bar, preferably from 3 to 35 bar. The molar ratio of phosgene to amino groups used is in general from 1.1:1 to 12:1, preferably from 1.25:1 to 8:1. The total residence time in the reactors is in general from 10 seconds to 15 hours, preferably from 3 min to 12 h. The reaction temperature is in general from 25 to 260° C. (degrees Celsius), preferably from 35 to 240° C.

The process according to the invention is suitable for the preparation of all conventional aliphatic and aromatic isocyanates, or a mixture of two or more such isocyanates. For example, monomeric methylene-di(phenyl isocyanate) (m-MDI) or polymeric methylene-di(phenyl isocyanate (p-MDI), toluene diisocyanate (TDI), R,S-1-phenylethyl isocyanate, 1-methyl-3-phenylpropyl isocyanate, naphthyl diisocyanate (NDI), n-pentyl isocyanate, 6-methyl-2-heptane isocyanate, cyclopentyl isocyanate, hexamethylene diisocyanate (HDI), isophorone diisocyanate (IPDI), diisocyanatomethylcyclohexane (H₆TDI), xylene diisocyanate (XDI), diisocyanatocyclohexane (t-CHDI) and di(isocyanatocyclohexyl)methane (H₁₂MDI) are preferred.

The process is particularly preferably used for the preparation of TDI, m-MDI, p-MDI, HDI, IPDI, H₆TDI, H12MDI, XDI, t-CHDI and NDI, in particular for the preparation of TDI.

The process according to the invention comprises continuous, semicontinuous and batchwise processes. Continuous processes are preferred.

The preparation of the isocyanates is usually effected by reacting the corresponding primary amine with an excess of phosgene. This process preferably takes place in the liquid phase.

An additional inert solvent may be added to the process according to the invention. This additional inert solvent is usually an organic solvent or mixtures thereof.

Chlorobenzene, dichlorobenzene, trichlorobenzene, toluene, hexane, diethyl isophthalate (DEIP), tetrahydrofuran (THF), dimethylformamide (DMF), benzene and mixtures thereof are preferred. Contrary to numerous publications in the prior art, the isocyanate prepared in the plant cannot be used as a solvent. Chlorobenzene is particularly preferred as the solvent.

The amine content, based on the amine/solvent mixture, is usually from 1 to 50% by mass, preferably from 2 to 40% by mass, particularly preferably from 3 to 30% by mass.

After the reaction, the mixture of substances is preferably separated into isocyanate(s), solvent, phosgene and hydrogen chloride by means of rectification. Small amounts of byproducts which remain in the isocyanate(s) can be separated from the desired isocyanate(s) by means of additional rectification or crystallization.

Depending on the choice of the reaction conditions, the product may contain inert solvent, carbamoyl chloride and/or phosgene and can be further processed by the known methods,

In the reaction, the phosgene first reacts with the amino groups to give carbamoyl chloride with elimination of hydrogen chloride. The carbamoyl chloride group then reacts with further elimination of hydrogen chloride to give the isocyanate group.

After the end of the reaction, the hydrogen chloride formed and the excess phosgene are usually separated off from the reaction mixture by distillation or by stripping with an inert gas. The hydrogen chloride/phosgene mixture is usually separated into hydrogen chloride and phosgene by distillation (FR 1 469 105) or by scrubbing with a hydrocarbon, the cost of the separation of HCl and phosgene being determined by purity requirements for the HCl and/or for the phosgene. Here, a distinction must be made between the content of phosgene in the HCl and the content of HCl in the phosgene. The phosgene thus obtained and freed from HCl is mixed with fresh phosgene from the phosgene synthesis and recycled to the reaction for the preparation of the isocyanate.

Depending on the mode of operation of the plant, the phosgene-containing stream which is fed to the reaction or mixing apparatus contains not only phosgene and the proportions of HCl mentioned but also the solvent in which the phosgenation is carried out. This is the case in particular when the separation of the phosgene and the hydrogen chloride is carried out by means of scrubbing with the solvent.

The invention furthermore relates to a production plant which is suitable for carrying out the process according to the invention. A preferred embodiment of a production plant according to the invention comprises the apparatuses I, II, III, V, VI and, if appropriate, the apparatuses VII, VIII, IX and IX according to FIG. 1. It is essential that the phosgene present in the phosgenation vessel I
(i) have a mass content of isocyanate of from 0.00001 to 1%, preferably from 0.0001% to less than 0.5% by mass, more preferably from 0.001% to less than 0.3% by mass, particularly preferably from 0.01% to less than 0.2% by mass, and
(ii) have a mass content of hydrogen chloride of from 0.00001% to less than 0.4% by mass, preferably from 0.0001% to less than 0.3% by mass, particularly preferably from 0.0005% to less than 0.25% by mass and very particularly preferably from 0.001% to less than 0.2% by mass.

The process according to the invention thus has the advantage that the number of shutdowns for cleaning the nozle and hence the number of plant downtimes can be considerably reduced compared with the known processes, For cleaning the nozzle, the plant usually has to be made phosgene-free and then opened, At the same time, the operability of the plant was thus also improved by reducing the openings. The technical effect of the process according to the invention is particularly surprising because, during the reaction for the formation of isocyanate, many times the amount of hydrogen chloride introduced by recycling the excess phosgene into the process is formed.

### EXAMPLE 1 ACCORDING TO THE INVENTION

A toluylene solution stream and a phosgene solution stream were mixed in the coaxial double-tube mixing nozzle of a pilot plant The toluenediamine solution stream of 5 kg/h, which consisted of 85% by weight of monochlorobenzene (MCB) and of 15% by weight of toluenediamine (TDA), the TDA consisting of 80% by weight of 2,4-TDA and of 20% by weight of 2,6-TDA, were sprayed in via the inner tube at a velocity of 7 m/s. The phosgene solution stream of 6.78 kg/h was fed in via the outer annular gap at a velocity of 5.8 m/s. The phosgene solution stream contains 0.02% by weight of hydrogen chloride (HCl) in addition to 90% by weight of phosgene and at least 9% by weight of MOB. The temperatures of the streams of starting materials were adjusted so that the stream emerging from the mixing nozzle had a temperature of 150° C. This stream was then passed through a stirred kettle with a residence time of 20 min. The pressure in the stirred kettle was 10 bar abs. The liquid reaction discharge was passed via a level control valve, and the gaseous reaction discharge was passed via a pressure control valve, onto a column in which a phosgene-containing stream and hydrogen chloride-containing stream were distilled off. The column operated at a pressure of 3.5 bar abs. The plant was operated for 72 hours without blockage problems.

### COMPARATIVE EXAMPLE 2

As in example 1, a toluenediamine solution stream and a phosgene solution stream were mixed in the coaxial double-tube mixing nozzle of a pilot plant. The toluenediamine solution stream of 5 kg/h, which consisted of 85% by weight of monochlorobenzene (MCB) and of 15% by weight of toluenediamine (TDA), the TDA consisting of 80% by weight of 2,4-TDA and of 20% by weight of 2,6-TDA, was sprayed in via the inner tube at a velocity of 7 m/s. The phosgene solution stream of 6.78 kg/h was fed in via the outer annular gap at a velocity of 5.8 m/s. The phosgene solution stream also contained 0.70% by weight of hydrogen chloride (HCl) in addition to 90% by weight of phosgene and at least 9% by weight of MCOB The temperatures of the streams of starting materials were adjusted so that the stream emerging from the mixing nozzle had a temperature of 150° C. This stream was then passed through a stirred kettle with a residence time of 20 min. The pressure in the stirred kettle was 10 bar abs. The liquid reaction discharge was passed via a level control valve, and the gaseous reaction discharge was passed via a pressure control valve, onto a column in which a phosgene-containing stream and hydrogen chloride-containing stream were distilled off. The column operated at a pressure of 3.5 bar abs. After 3.6 h, the plant had to be shut down owing to blockage problems in the level control valve of the stirred kettle.

1. A process for the preparation of isocyanates by reacting amines with phosgene, the phosgene-containing stream of starting materials
(i) being substantially free of isocyanates and
(ii) having a mass content of hydrogen chloride of less than 0.4% by mass.

2. The process according to claim 1, which is carried out according to FIG. 1.

3. The process according to claim 1, wherein the phosgene-containing stream of starting materials has a mass content of hydrogen chloride of from 0.00002% to 0.3%.

4. The process according to claim 1, wherein the phosgene-containing stream of starting materials is mixed with an amine-containing stream of starting materials in a mixing time of from 0.0001 second to 5 seconds.

5. The process according to claim 1, which is used for the preparation of an isocyanate selected from the group consisting of TDI, m-MDI, p-MDI, HDI, IPDI, H6TDI, H12MDI, XDI, t-CHDI and NDI.

6. The process according to claim 1, wherein the reaction is carried out in a temperature range from 25 to 260° C. and at absolute pressures of from 0.9 bar to 400 bar, the molar ratio of phosgene to amino groups used being from 1.1:1 to 12:1.

7. The process according to claim 1, wherein said process is carried out by a continuous method and the reaction of phosgene with amine is effected in the liquid phase.

8. The use of phosgene, the phosgene being substantially free of isocyanates and having a mass content of hydrogen chloride of less than 0.4% by mass, for the preparation of isocyanates by phosgenation of primary amines.

9. A production plant for the preparation of isocyanates by reacting primary amines with phosgene, comprising an amine vessel, a phosgene vessel, a mixing apparatus, a reactor and a working-up apparatus, wherein the phosgene present in the phosgene vessel
(i) has a mass content of isocyanate of from 0.00001 to 1% and
(ii) a mass content of hydrogen chloride of from 0.00001% to less than 0.4% by mass.
